(19) United States
(12) Patent Application Publication
(10) Pub. No.: US 2023/0363955 A1
(43) Pub. Date: Nov. 16, 2023

(54) Dissector/Absorber Device for Surgical Applications

(71) Applicant: Ebrahim Elahi, New York, NY (US)

(72) Inventor: Ebrahim Elahi, New York, NY (US)

(21) Appl. No.: 18/144,894

(22) Filed: May 9, 2023

Related U.S. Application Data
(60) Provisional application No. 63/340,195, filed on May 10, 2022.

(51) Int. Cl. A61F 13/38; A61B 17/32

(52) U.S. Cl. CPC A61F 13/38; A61B 17/32; A61B 2017/320044

(57) ABSTRACT

According to at least one exemplary embodiment, a single handheld medical dissector/absorber device performs both absorption and dissection during a surgical procedure. Such a single medical device may include an elongate handle having both an absorber component and dissector component attached at one end. The absorber and dissector components (or regions or areas) may be partially or fully concentric with respect to each other, such that a first portion of the dissector is located concentrically within the absorber, while a second portion of the dissector protrudes out of the end portion of the absorber. In operation, as the protruding second portion of the dissector performs a dissection of, for example, tissue, the absorber absorbs any fluid (e.g., blood) concurrently as the dissection is occurring.

This application claims the benefit under 35 U.S.C 119 from provisional application no. 63/340,195, filed May 10^{th}, 2022, incorporated by reference herein in its entirety.

### BACKGROUND

The present disclosure generally relates to medical devices, and particularly to medical handheld devices used to assist with surgical procedures.

For surgical procedures, currently there are highly absorbent sponges. However, these sponges may not provide the friction necessary to dissect, which involves the ability to create shearing forces (blunt dissection) and, for example, accordingly, manipulate tissue. Products that do indeed provide shearing forces (peanuts, cotton tips and rosebud dissectors) are, however in contrast, generally poor at absorbing, which leads to having to use a lot of irrigation, cottonoid sponges, or other means of obtaining hemostasis. Thus, the use of separate devices, one to perform absorption and another to perform dissection, may lead to surgical inefficiency and risk, especially when it comes to surgery in small spaces (neurosurgery, ENT, ophthalmology etc.).

### BRIEF SUMMARY

According to at least one exemplary embodiment, a single handheld medical dissector/absorber device performs both absorption and dissection concurrently during a surgical procedure. Such a single medical device may include an elongate handle having both an absorber component and dissector component attached at one end. The absorber and dissector components (or regions or areas) may be partially or fully concentric with respect to each other, such that a first portion of the dissector is located concentrically within the absorber, while a second portion of the dissector protrudes out of the end portion of the absorber. In operation, as the protruding second portion of the dissector performs a dissection of, for example, tissue, the absorber absorbs any fluid (e.g., blood) concurrently as the dissection is occurring.

According to at least one other exemplary embodiment, a single handheld medical dissector/absorber device performs both absorption and dissection concurrently during a surgical procedure. Such a single medical device may include an elongate handle having both an absorber component and dissector component attached at one end. The absorber and dissector components (or regions or areas) may be partially or fully concentric with respect to each other, such that the absorber is located concentrically within the dissector. In operation, as the dissector performs a dissection of, for example, tissue, the concentric inner absorber absorbs any fluid (e.g., blood) concurrently as the dissection is occurring.

### BRIEF DESCRIPTION OF THE SEVERAL VIEWS OF THE DRAWINGS

FIG. 1 illustrates an exemplary medical dissector/absorber device according to one embodiment.

FIG. 2 illustrates an exemplary embodiment of a medical dissector/absorber device like that depicted in FIG. 1 that further includes squeezing tongs for removing liquid absorbed by the absorber component, region, or area.

FIG. 3 illustrates an exemplary embodiment of a medical dissector/absorber device similar to that depicted in FIG. 1, where a hexagonal handle coupling portion couples the handle portion of the device to an absorber/dissector head-changing tray.

FIG. 4 illustrates an exemplary embodiment of a medical dissector/absorber device similar to that depicted in FIG. 1, whereby the handle portion and the absorber/dissector component of the device are a single unit or piece.

FIG. 5 illustrates another exemplary embodiment of a medical dissector/absorber device that includes an absorber and dissector that are concentric with respect to one another having a single piece handle and multiple absorption characteristics.

FIG. 6 illustrates an exemplary embodiment of a medical dissector/absorber device that includes an absorber and dissector, where a portion of the absorber extends into a cavity region of the handle portion of the device.

FIG. 7 illustrates an exemplary embodiment of a medical dissector/absorber device that is similar to that of FIG. 6, except for an actuating lever located on the handle portion instead of having a suction mechanism.

FIG. 8 illustrates an exemplary embodiment of a medical dissector/absorber device that is similar to that of FIG. 7, except for perforations/holes located on the handle portion.

FIG. 9 illustrates yet another exemplary embodiment of a medical dissector/absorber device, where the dissector and absorber regions are adjacent as opposed to in a concentric or partially concentric relationship.

FIG. 10 illustrates yet another exemplary embodiment of a medical dissector/absorber device similar to that of FIG. 9, except the protruding region can be further manipulated using an absorber applicator.

FIG. 11 illustrates yet another exemplary embodiment of a medical dissector/absorber device similar to that of FIG. 10, except, here, the dissector is adhered directly to the absorber region.

FIG. 12 illustrates yet another exemplary embodiment of a medical dissector/absorber device, where the dissector is adhered directly to the absorber region, and the absorber region is adhered to a flexible stem handle having a grip such as a thumb and index finger grip.

FIG. 13 illustrates yet another exemplary embodiment of a medical dissector/absorber device similar to that of FIG. 12, except an additional raised portion (i.e., an additional pressure point) is incorporated for controlling the flexing of the handle.

FIG. 14 illustrates yet another exemplary embodiment of a medical dissector/absorber device similar to that of FIG. 12, where the absorber region includes two absorbing fins.

FIG. 15 illustrates yet another exemplary embodiment of a medical dissector/absorber device similar to that of FIG. 12, where the absorber region includes four absorbing fins.

### DETAILED DESCRIPTION

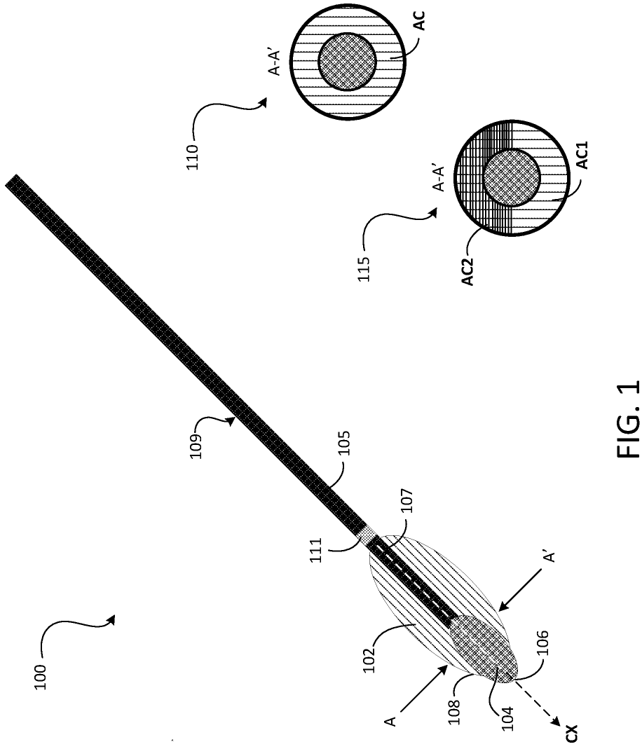

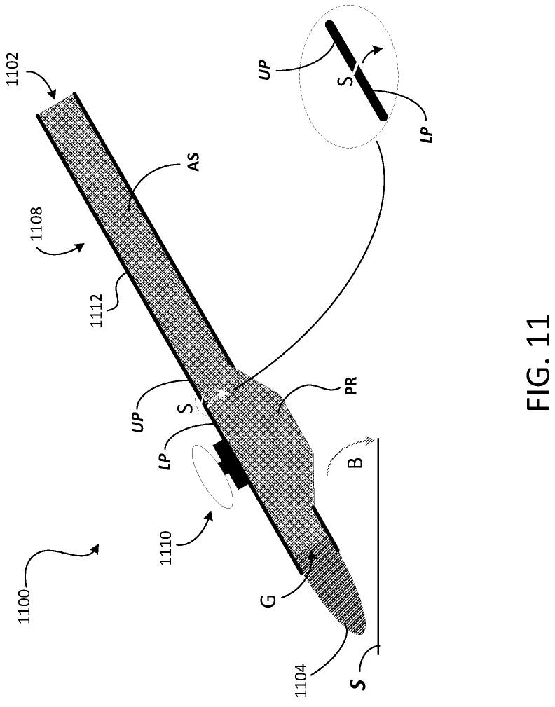

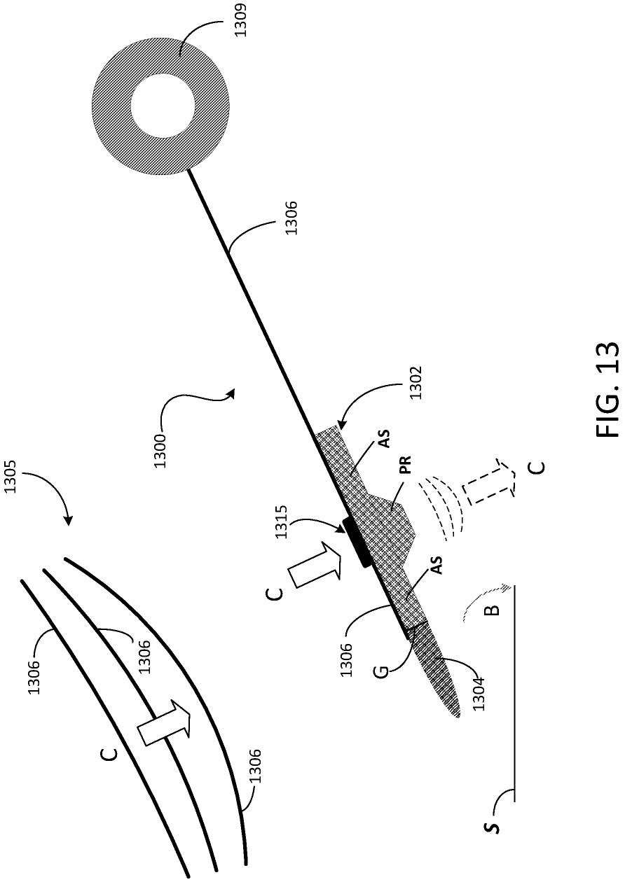

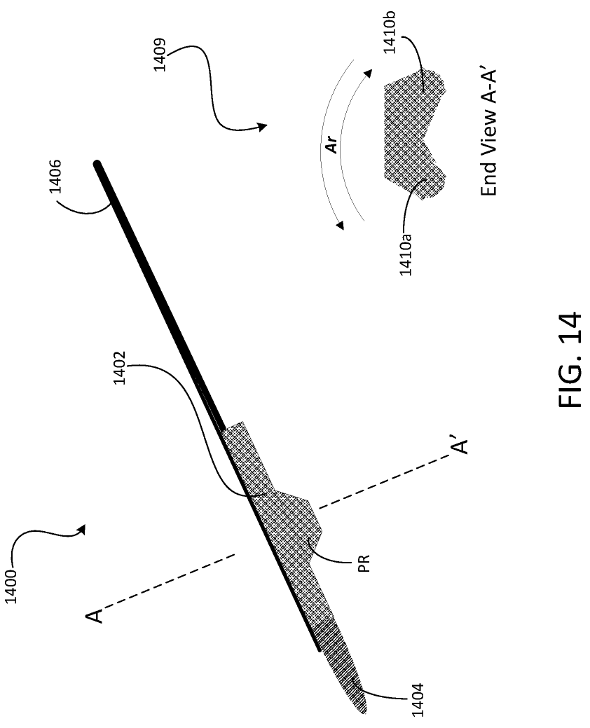

Accordingly, the above-described embodiments associated with FIGS. 1-15 include various features and applications, which can be interchangeably applied to each embodiment. For example, the thumb and index finger grip shown in FIG. 13 can be utilized by the exemplary embodiment of FIG. 14, or FIG. 1. In some embodiments, the absorber and dissector components are described as being directly adhered to each other either in the concentric configuration (e.g., FIG. 1) or adjacent configuration (FIG. 11). However, it can be contemplated that these components may be adhered to each other with one or more interface layers between them. The one or more interface layers (e.g., plastic or silicone layer(s)) can isolate the travel or liquid between the absorber and dissector components, or in some instances, facilitate a predetermined transference of liquid between the components.

In terms of dimensionality, the described and illustrated embodiments can be dimensionally scaled in accordance with the type of surgery being carried out. For example, dissector/absorber devices used for ophthalmology applications are going to be relatively smaller than those used for surgical procedures on larger anatomical organs.

The concurrent absorption and dissection referred to herein refers to any instant in time where, as tissue or other matter is being dissected, any resulting accumulated blood and/or other fluid is simultaneously absorbed.

Different materials can be used for the absorber and dissector components. The absorber’s absorbing properties can include PVA synthetic sponges that are composed of Polyvinyl Alcohol, which can include a wide arrange of pore sizes and water holding capacity. For example, characteristics such as pores per centimeter (PPC), density, smoothness, solids penetration, water absorption rate, abrasion resistance, squeezability (ease of water expulsion), water release speed, etc., are but some absorbing properties for an absorber. Absorber sponge technologies for medical applications are manufactured by companies such as RAMERFOAM TECHNOLOGY®. The dissector can be formed from smooth spun cotton (e.g., sterile %100 cotton). Such dissector materials can be found on ENDOSTIK® endoscopic sponges supplied by FABCO®. Example materials used for the dissector may include hydroxylated polyvinyl acetal.

FIG. 1 illustrates an exemplary embodiment of a medical dissector/absorber device 100 that includes an absorber 102 and dissector 104 oriented concentrically (i.e., sharing the same center axis CX) with respect to one another. As illustrated, the end portion of the dissector 104 partially protrudes beyond the absorber 102 component to facilitate the sheering needed for the dissection operation.

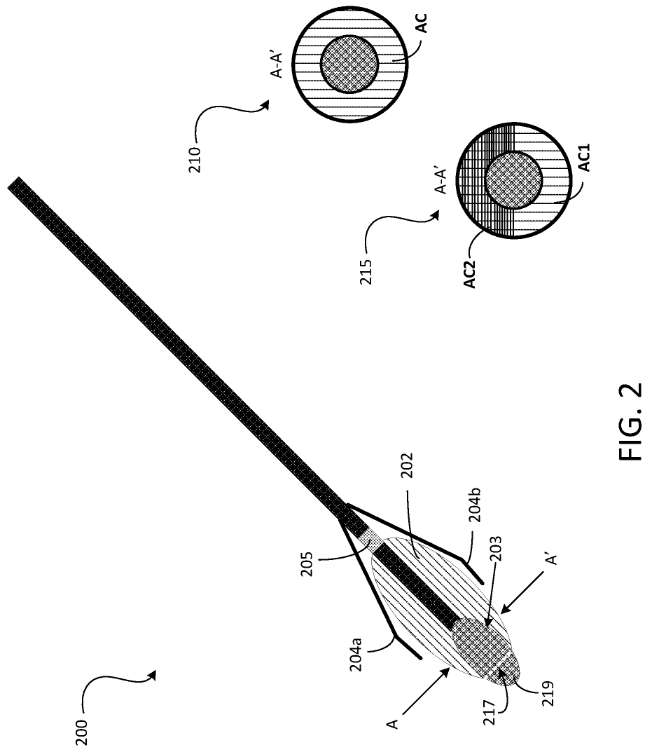

As with FIG. 1, FIG. 2 also shows the end portion 219 of the dissector 203 protruding beyond the end 217 portion of the absorber 202. The amount by which the end portion 219 of the dissector 203 protrudes beyond the end 217 portion of the absorber 202 may vary by surgical application. In some embodiment, the protrusion of the end portion of the dissector 203 may be between about 20%-50% (±5%) of the overall dissector length. For example, if the dissector’s 203 total length is 20 millimeters (mm), a protrusion of 20% would mean the end portion 217 is 4 mm and the remaining portion of the dissector encompassed by the absorber 202 is 16 mm.

Referring back to FIG. 1, as blood and/or other fluid passes over and/or alongside the protruding dissector region 106 during the dissecting process, it is captured and absorbed by the absorption properties of the absorber component 102. According to some embodiments, the end 106 of the dissector 104 may be approximately flush with the outer periphery 108 of the absorber 102 and thus not protrude or protrude less.

The medical dissector/absorber device 100 may include a handle 109 that includes a detachable handle portion 105 and a head portion 107 to which the absorber 102 and dissector 104 are coupled. The head portion 107 may be detached from, or attached to, the detachable handle portion 105 via a screw or a snap fit coupling 111. This allows for the replacement of the head portion 107 when the absorber 102 and/or the dissector 104 become worn to the point where a user/physician/surgeon determines that a new absorber 102 and/or the dissector 104 is needed. In such a scenario, the head portion 107 including the worn absorber 102 and/or worn dissector 104 may be replaced with a head portion 107 including a new absorber 102 and a new dissector 104. According to some embodiments, the handle 109 may be contemplated as a single elongate component to which the absorber 102 and dissector attach in the manner described. In this scenario, as the absorber 102 and/or dissector 104 become worn, the entire dissector/absorber device 100 is disposed of and replaced.

As depicted in FIG. 1, the dissector 104 may be adhered, at least partially, over the end portion of the head portion 107, while the absorber 102 may be adhered over both the dissector 104 and a portion of the head portion 107.

A cross section along A-A′ of the dissector/absorber device 100 is depicted according to two different examples, as shown by 110 and 115. According to example cross section 110, the absorber 102 includes a single absorption characteristic AC. However, according to example cross section 115, the absorber 102 may include multiple absorption characteristic regions having, for example, a first absorption characteristic AC1 and a second absorption characteristic AC2 that is different than the first absorption characteristic AC1.

In operation, based on the amount of fluid (e.g., blood) present for absorption, the dissector/absorber device 100 can be manipulated by a user/physician/surgeon to bring a lower absorption region (e.g., AC1) in contact with the fluid during the dissecting procedure. However, based on a relative increase in the among of fluid (e.g., blood) present for absorption, a user/physician/surgeon may rotate the detachable handle portion 105 of the dissector/absorber device 100 to now bring a higher absorption region (e.g., AC2) in contact with the increased fluid amount during the dissecting procedure.

FIG. 2 illustrates an exemplary embodiment of a medical dissector/absorber device 200 that is similar to that of FIG. 1. As depicted in FIG. 2, compared to the medical dissector/absorber device 100 of FIG. 1, medical dissector/absorber device 200 includes additional squeezing tongs 204a, 204b for removing liquid absorbed by the absorber 202. The medical dissector/absorber device 200 is thus identical to that of the medical dissector/absorber device 100 of FIG. 1, except for the squeezing tongs 204a, 204b that are configured to dispense absorbed liquid from the absorber 202 upon actuation by a user/physician/surgeon. The actuation of the tongs 204a, 204b is configured to compress the absorber 202 to the extent that absorbed liquid is squeezed out, thus allowing for subsequent fluid absorption by the absorber 202.

A cross section along A-A′ of the dissector/absorber device 200 is depicted according to two different examples, as shown by 210 and 215. According to example cross section 210, the absorber 202 includes a single absorption characteristic AC. However, according to example cross section 215, the absorber 202 may include multiple absorption characteristic regions having, for example, a first absorption characteristic AC1 and a second absorption characteristic AC2 that is different than the first absorption characteristic AC1.

In operation, based on the among of fluid (e.g., blood) present for absorption, the dissector/absorber device 200 can be manipulated by a user/physician/surgeon to bring a lower absorption region (e.g., AC1) in contact with the fluid during the dissecting procedure. However, based on a relative increase in the among of fluid (e.g., blood) present for absorption, a user/physician/surgeon may rotate the detachable handle portion 205 of the dissector/absorber device 200 to now bring a higher absorption region (e.g., AC2) in contact with the increased fluid amount during the dissecting procedure. Upon actuation of the squeezing tongs 204a, 204b by a user/physician/surgeon, absorbed liquid is dispended from one or more regions (e.g., AC or AC1 and AC2) of absorber 202 based on the number of absorption regions incorporated within the absorber 202.

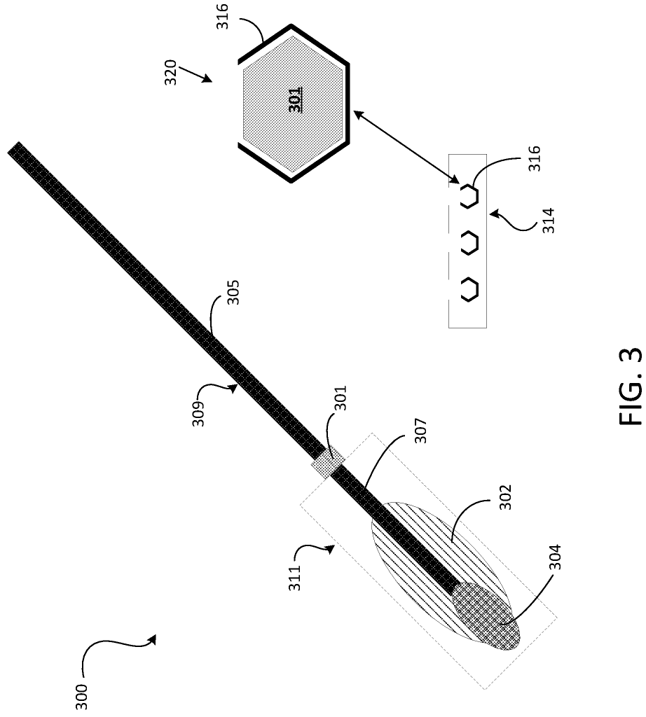

FIG. 3 illustrates an exemplary embodiment of a medical dissector/absorber device 300 similar to that depicted in FIG. 1, except a coupling mechanism such as a shaped (e.g., hexagonal) handle coupling portion 301 couples the handle portion 309 of the device 300 to an interchangeable/replaceable lower portion (as indicated by 311) of the device 300 that includes the absorber 302, dissector 304, and head portion 307.

In operation, an absorber/dissector head-changing tray 314 (shown as a plan view) will retain the interchangeable/replaceable lower portion 311 of the device 300 using a hexagonal receptacle 316 configured to receive the hexagonal handle coupling portion 301. This is further illustrated using expanded-view 320, whereby the hexagonal handle coupling portion 301 is placed in and retained by the hexagonal receptacle 316 during a rotation movement of a detachable handle portion 305 of the handle 309. A user/physician/surgeon is thus able to screw-in or snap-in the detachable handle portion 305 of the handle 309 by rotating portion 305 into the head portion 307 of interchangeable/replaceable lower portion 311 while the interchangeable/replaceable lower portion 311 is retained by the hexagonal receptacle 316 of head-changing tray 314.

Similarly, the hexagonal profile of the hexagonal receptacle 316 of head-changing tray 314 and the hexagonal shape of the example coupling mechanism 301 can facilitate rotating the handle portion 305 out of the head portion 307 part of the interchangeable/replaceable lower portion 311 by keeping this lower portion 311 component stationary during the rotation of the handle portion 305 out of the head portion 307. This subsequently provides a decoupling of the handle portion 305 from the interchangeable/replaceable absorber 302 and dissector component 304 of the interchangeable/replaceable lower portion 311.

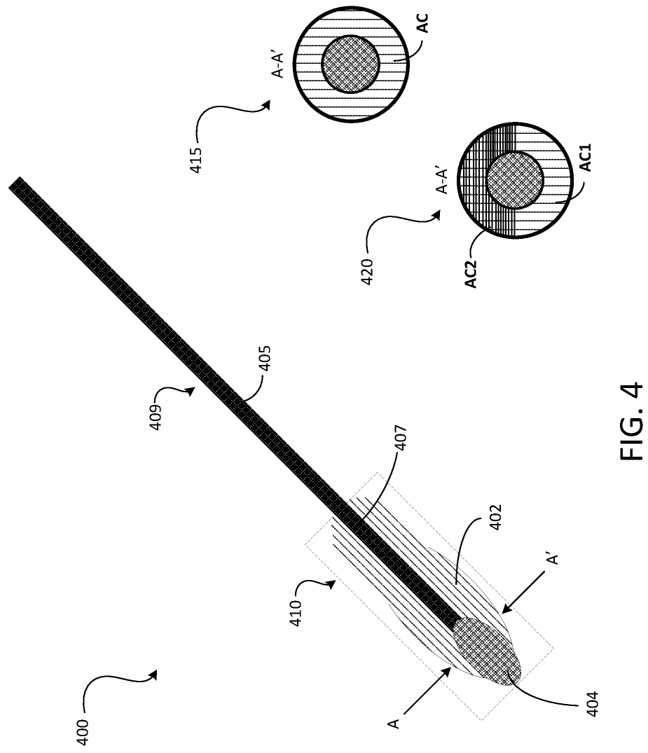

FIG. 4 illustrates an exemplary embodiment of a medical dissector/absorber device 400 similar to that depicted in FIG. 1, whereby the handle portion 405 and head portion 407 of the device handle 409 is a single uniform piece without a coupling mechanism 301 separating them such as that shown in FIG. 3. According to this embodiment, and as referred to in relation to the description of FIG. 1, the handle 409 may be contemplated as a single elongate component to which the absorber 402 and dissector 404 attach. In this scenario, as the absorber 402 and/or dissector 404 become worn, the entire dissector/absorber device 400 can be disposed of and replaced.

A cross section along A-A′ of the dissector/absorber device 400 is depicted according to two different examples, as shown by 415 and 420. According to example cross section 415, the absorber 402 includes a single absorption characteristic AC. However, according to example cross section 420, the absorber 402 may include multiple absorption characteristic regions having, for example, a first absorption characteristic AC1 and a second absorption characteristic AC2 that is different than the first absorption characteristic AC1.

In operation, based on the among of fluid (e.g., blood) present for absorption, the dissector/absorber device 400 can be manipulated by a user/physician/surgeon to bring a lower absorption region (e.g., AC1) in contact with the fluid during the dissecting procedure. However, based on a relative increase in the among of fluid (e.g., blood) present for absorption, a user/physician/surgeon may rotate the detachable handle portion 405 of the dissector/absorber device 400 to now bring a higher absorption region (e.g., AC2) in contact with the increased fluid amount during the dissecting procedure.

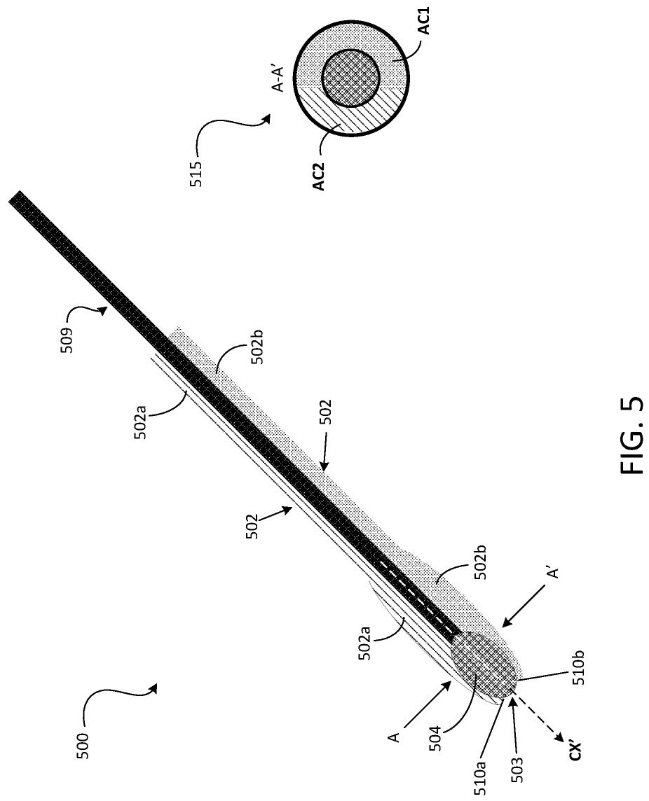

FIG. 5 illustrates an exemplary embodiment of a medical dissector/absorber device 500 that includes an absorber 502 and dissector 504 that are concentric (i.e., having a common center axis CX′) with respect to one another. As depicted, the absorber may include a plurality (e.g., two) of absorber regions 502a, 502b each having different absorbing characteristics. Compared to other disclosed exemplary embodiments (e.g., FIGS. 1-5), the dissector 504 tip 503 may be substantially flush with the ends 510a, 510b of the absorber regions 502a, 502b, respectively, such that the dissector 504 tip 503 does not protrude, or protrudes very little beyond the absorber regions 502a, 502b. Moreover, the absorber region extends along a greater portion of the device handle 509.

In operation, initially, a surgeon may rotate the handle 509 to bring the portion of the absorber having the desired absorbing property (e.g., region 502a having higher absorption) in contact with the fluid they wish to absorb. Subsequently, the surgeon may then rotate the handle 509 to bring the other portion of the absorber having a different absorbing property (e.g., region 502b having lower absorption) in contact with the fluid they wish to further absorb. Accordingly, to facilitate this, the different portions of the absorber having different absorption characteristics may, for example, include different colors to signify the difference between the absorbing properties.

A cross section along A-A′ of the dissector/absorber device 500 is depicted, as shown by 515. Accordingly, absorber region 502a may include one absorption characteristic AC1 while absorber region 502b may include another absorption characteristic AC2 that is different than the first absorption characteristic AC1.

While FIGS. 1-5 illustrate an exemplary device embodiment having a concentric relationship between the absorber and dissector, a partially concentric relationship between the absorber and dissector may also be contemplated, whereby the absorber partially wraps around the dissector as opposed to entirely wrapping around the dissector at at-least one circumferential portion.

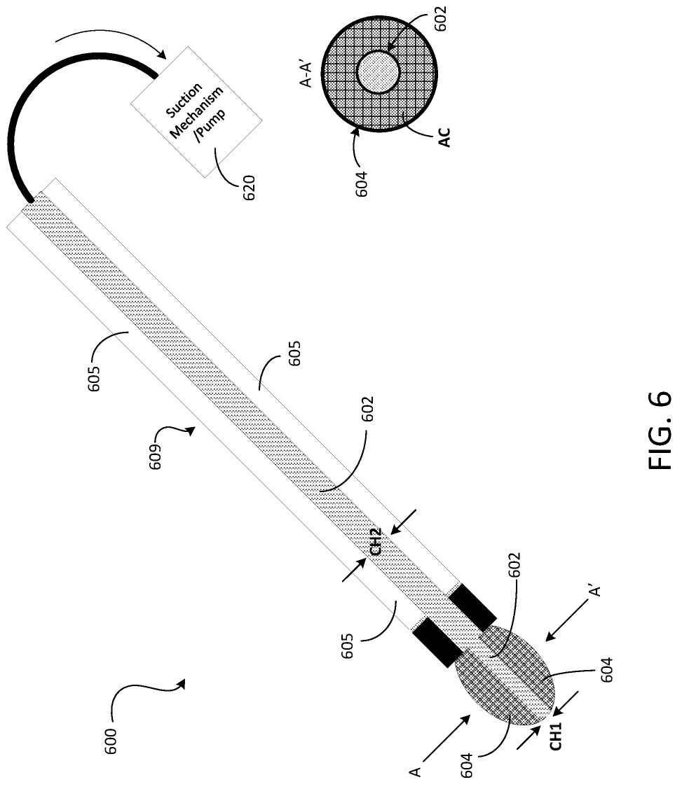

FIG. 6 illustrates an exemplary embodiment of a medical dissector/absorber device 600 that includes an absorber 602 and dissector 604 that are concentric with respect to one another. In contrast to the prior disclosed embodiments, here, the absorber 602 forms the inner concentric portion, while the dissector 604 is the outer region of the concentric relationship between the absorber 602 and dissector regions 604.

The inner absorber 602 also further extends into a cavity region 605 of the handle portion 609 of the device, thus facilitating an increased absorption of liquid relative to if the device did not have such an extended absorber region within the handle 609. This can be particularly instrumental based on the relatively narrow channel width CH1 the absorber 602 forms at the tip portion of the absorber/dissector 600 relative to portion of the absorber 602 located within the cavity region 605 of the handle portion 609 (i.e., channel width CH2).

It may be appreciated that multiple absorption channels (e.g., two or more) and patterns of absorber channel (e.g., different channel width ratios, CH1/CH2, tapered channel cross-sections, etc.) associated with the absorber 602 can be comprehended based on the surgical process and the needs for absorbing and dissecting during a surgical procedure. As further depicted, a suction mechanism 620 can enable the removal of absorbed liquid from the absorber 602.

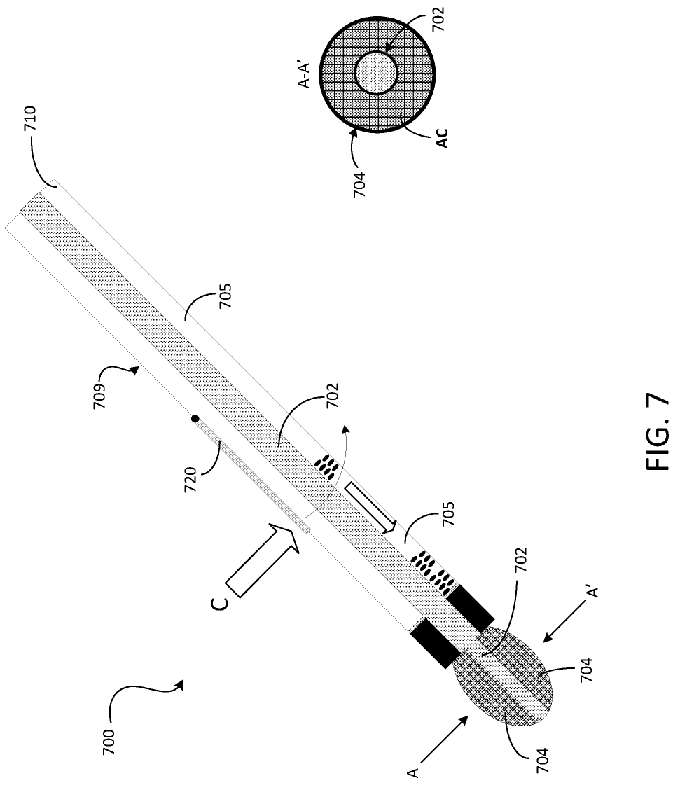

FIG. 7 illustrates an exemplary embodiment of a medical dissector/absorber device 700 having an absorber 702 and dissector 704 that is identical to that of FIG. 6, except for an actuating lever 720 located on and coupled to the handle portion 709, instead of having a suction mechanism 620 (FIG. 6). Here, by actuating the actuating lever 720 in direction C, the region of the absorber 702 under the actuating portion is compressed, releasing the absorbed liquid within the absorber 702 into the cavity region 705 of the handle portion 709. Subsequently, the collected liquid (e.g., blood) within the cavity 705 of the handle 709 can be dispensed via opening 711 located at the top of the handle 709.

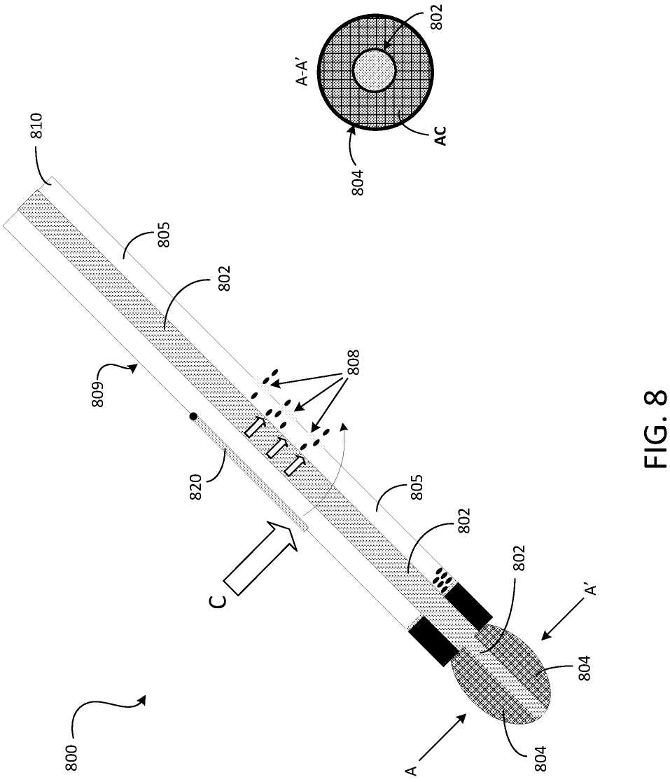

FIG. 8 illustrates an exemplary embodiment of a medical dissector/absorber device 800 having an absorber 802 and dissector 804 that is identical to that of FIG. 7, except for perforations/holes 808 located on the handle portion 809. Here, by actuating the actuating lever 820 in direction C, the region of the absorber 802 under the actuating portion is compressed, releasing the absorbed liquid from the absorber both into the cavity region 805 of the handle 809 and through the perforations 808 on the handle portion. Subsequently, the collected liquid (e.g., blood) within the handle portion cavity 805 can be dispensed in the same manner as with that described in relation to the embodiment of FIG. 7. The perforations 808 or openings facilitate a liquid dispensing means that enables such a liquid-dispensing during the dissecting operation without the need to expense the liquid through the end 810 of the handle 809, thus avoiding an interruption of a surgical operation during use of the device 800.

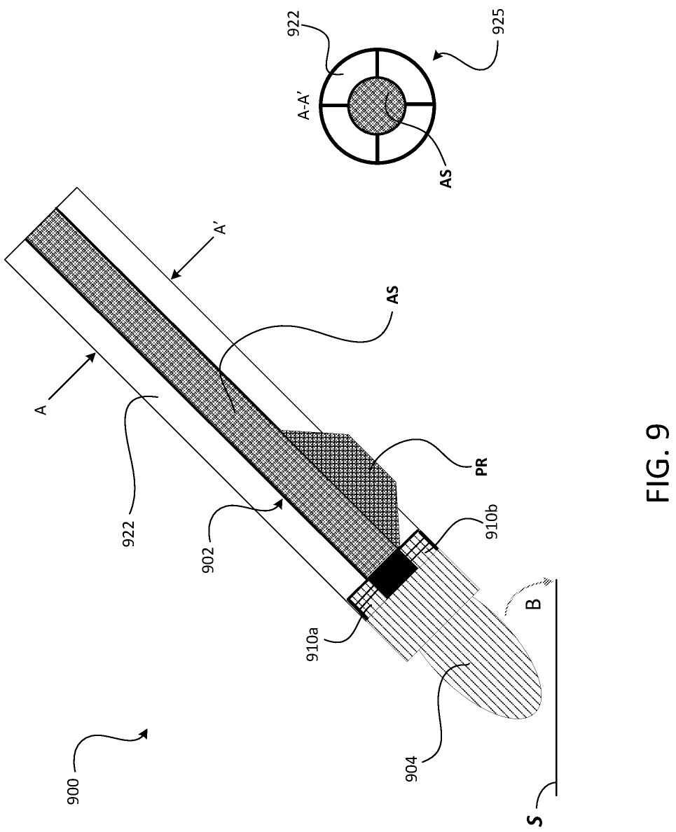

FIG. 9 illustrates yet another exemplary embodiment of a medical dissector/absorber device 900, where the dissector 904 and absorber 902 regions are adjacent as opposed to in a concentric or partially concentric relationship. In the prior described embodiments, the absorbing and dissecting operations can be concurrently occurring, while here, the dissecting operation typically occurs first, before the absorbing of liquid (e.g., blood, etc.), which follows. More specifically, the dissector 904 at the tip of the device is adapted to dissect and parse tissue. The absorber 902 is housed within a cavity opening 922 of the handle portion 908 of the device 900, while the dissector 904 is coupled via attachment slots 910a, 910b to the end of the handle portion 908.

As blood or other fluid collects, the adjacent absorber 902, mainly the protruding region (PR) of the absorber 902, can be brought into contact with the collected blood on surface S by angularly manipulating the device in the direction of arrow B. As depicted, the protruding region (PR) and the absorber sponge (AS) of the absorber 902 collectively facilitate the absorption of blood and/or other fluid.

The housing of the absorber 902, in particular the absorption sponge (AS) within the cavity opening 922 of the handle portion 908 is further illustrated by the cross-section depiction 925 taken across A-A′. In some embodiments the protruding region (PR) and the absorber sponge (AS) of the absorber 902 include the same absorption characteristic (AC), while according to other embodiments, the protruding region (PR) and the absorber sponge (AS) of the absorber 902 may include different absorption characteristic (i.e., AC1 and AC2).

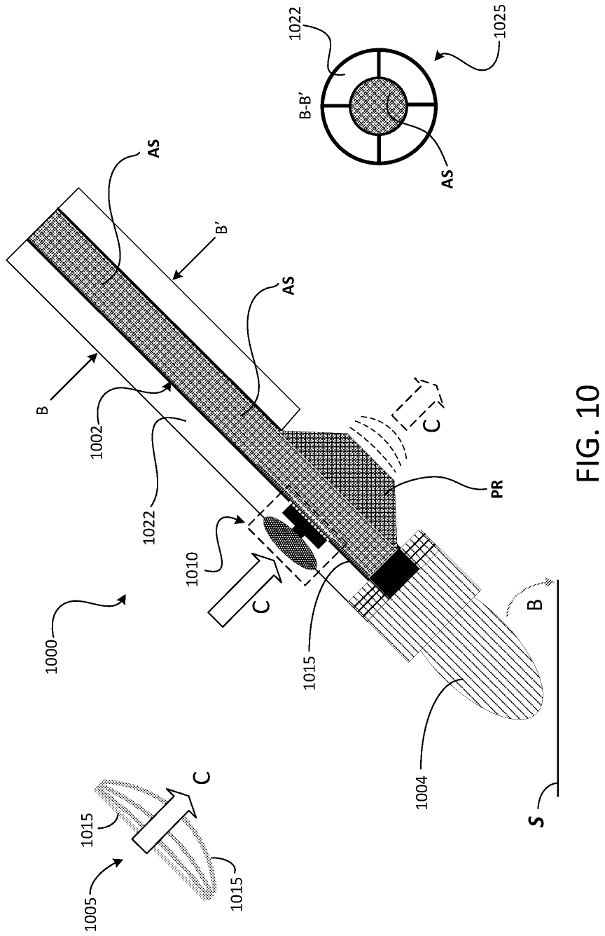

FIG. 10 illustrates yet another exemplary embodiment of a medical dissector/absorber device 1000 similar to that of FIG. 9, except the protruding region (PR) can be further manipulated using an absorber applicator 1010. As with the device of FIG. 9, here, the dissecting operation typically occurs first, followed by the absorbing of liquid (e.g., blood, etc.). More specifically, the dissector 1004 at the tip of the device is adapted to dissect and parse tissue. As blood or other fluid collects on surface S, the adjacent absorber 1002, particularly the protruding region (PR) of the absorber 1002, can be brought into contact with the collected blood by: (i) angularly manipulating the device in the direction of arrow B and/or (ii) actuating the absorber applicator 1010 in the direction of illustrative arrow C, thus causing the protruding region (PR) to further flex toward the surface S where the blood or fluid is or has collected. As depicted, the protruding region (PR) and the absorber sponge (AS) of the absorber 1002 collectively facilitate the absorption of blood and/or other fluid based on at least the dissection. As the protruding region (PR) makes contact with the blood or fluid, the absorber sponge (AS) with its greater volume provides the bulk of the fluid absorption function compared to the smaller protrusion region.

In order to facilitate the flexing of the protruding region (PR), the device 1000 includes a flex member 1015 that is located between (i.e., interfaced between) a region of the absorber sponge (AS) and the base of the absorber applicator 1010, as illustrated by the dashed white lines. The flex member 1015 may be a flexible elongate plastic that is adhered on its top side to the applicator 1010, while being adhered on its bottom side to the absorber sponge (AS). An expanded view of the illustrative flexing of the flex member based on actuating the absorber applicator 1010 in the direction of illustrative arrow Cis depicted at 1005.

The housing of the absorber 1002, in particular the absorption sponge (AS) within the cavity opening 1022 of the handle portion 1008 is further illustrated by the cross-section depiction 1025 taken across B-B′. In some embodiments the protruding region (PR) and the absorber sponge (AS) of the absorber 1002 include the same absorption characteristic (AC), while according to other embodiments, the protruding region (PR) and the absorber sponge (AS) of the absorber 1002 may include different absorption characteristic (i.e., AC1 and AC2).

FIG. 11 illustrates yet another exemplary embodiment of a medical dissector/absorber device 1100 similar to that of FIG. 10, except, here, the dissector 1104 is adhered directly to the absorber 1102 (i.e., at location G) as opposed to being formed as two separate components, as with the embodiment of FIG. 10. As with the device of FIG. 10, the dissecting operation typically occurs first, followed by the absorbing of liquid (e.g., blood, etc.). More specifically, the dissector 1104 at the tip of the device 1100 is adapted to dissect and parse tissue. As blood or other fluid collects, the adjacent absorber 1102, mainly the protruding region PR of the absorber 1102, can be brought into contact with the collected blood by: (i) angularly manipulating the device 1100 in the direction of arrow B and/or (ii) actuating the absorber applicator 1110 causing the protruding region PR to further flex toward the surface where the blood or fluid is or has collected. As the absorber applicator 1110 is actuated or pressed, an opening (i.e., at location S) in a rod section 1112, that forms part of the handle 1108 of the device, facilitates the detachment of the lower portion LP of the rod from its upper portion UP, allowing the lower portion LP of the rod to manipulate or flex the protrusion region PR of the absorber 1102 towards the surface S where the blood or fluid has accumulated or is accumulating.

As depicted, the protruding region (PR) and the absorber sponge (AS) of the absorber 1102 collectively facilitate the absorption of blood and/or other fluid based on at least the dissection. As the protruding region (PR) makes contact with the blood or fluid, the absorber sponge (AS) with its greater volume provides the bulk of the fluid absorption compared to the smaller protrusion region.

In some embodiments the protruding region (PR) and the absorber sponge (AS) of the absorber 1102 include the same absorption characteristic (AC), while according to other embodiments, the protruding region (PR) and the absorber sponge (AS) of the absorber 1102 may include different absorption characteristic (i.e., AC1 and AC2).

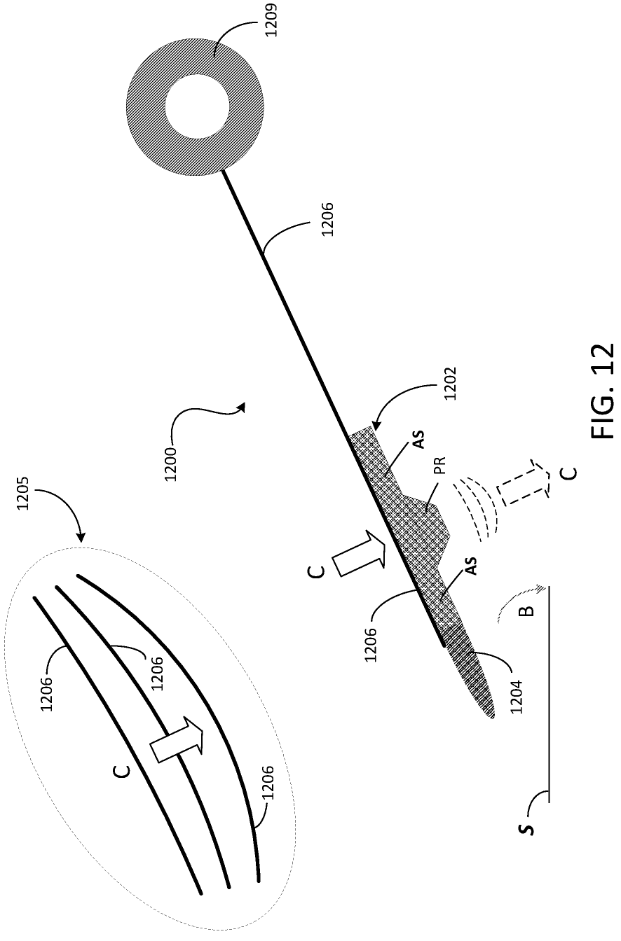

FIG. 12 illustrates yet another exemplary embodiment of a medical dissector/absorber device 1200, where the dissector 1204 is adhered directly to the absorber region 1202, and the absorber 1202 is adhered to a portion of a flexible stem handle 1206 having a grip such as a thumb and index finger grip 1209. As depicted, similar to that of FIGS. 9-11, the absorber region 1202 includes the protruding region PR for directly contacting the blood/fluid, and an absorber sponge AS for increasing the absorption capacity by absorbing the blood/fluid captured by the protruding region PR.

In operation, bending or flexing the flexible stem handle 1206 in the direction of arrow C facilitates an increased movement/manipulation of the protruding region PR of the absorber 1202 towards the surface S where blood or any other fluid may have collected. Thus, as blood or other fluid collects, the protruding region of the absorber can be brought into contact with the collected blood/fluid by: (i) angularly manipulating the device 1200 and/or (ii) flexing the flexible stem handle1206 (i.e., in the direction of arrow C) causing the protruding region PR to further flex toward the surface where the blood or fluid is or has collected. An illustrative flexing of the flexible stem handle 1206 in the direction of illustrative arrow C is depicted by 1205. In this embodiment, the thumb and index finger grip 1209 facilitates the manipulation of the device 1200 for dissecting and/or absorbing.

In some embodiments the protruding region (PR) and the absorber sponge (AS) of the absorber 1202 include the same absorption characteristic (AC), while according to other embodiments, the protruding region (PR) and the absorber sponge (AS) of the absorber 1202 include different absorption characteristic (i.e., AC1 and AC2).

FIG. 13 illustrates yet another exemplary embodiment of a medical dissector/absorber device 1300 similar to that of FIG. 12, except a raise portion 1315 (i.e., additional pressure point) is incorporated for controlling the flexing of the handle. Device 1300 includes an absorber 1302 and a dissector 1304 that are adhered directly to each other, as indicated at G. The absorber 1302 is adhered to a flexible stem handle 1306 having a grip such as a thumb and index finger grip 1309. As depicted, similar to that of FIGS. 9-12, the absorber region 1302 includes the protruding region PR for directly contacting the blood/fluid, and an absorber sponge AS for increasing the absorption capacity by absorbing the blood/fluid captured by the protruding region PR. While the protruding region PR is located on the bottom region of the absorber 1302, the raised portion 1315 is located opposite to the protruding region PR and adhered either directly to the opposing top region of the absorber 1302 or adhered to the flexible stem handle 1306 to which the absorber 1302 is adhered (i.e., the flexible stem handle 1306 is between the raised portion 1315 and absorber 1302).

In operation, bending or flexing the flexible stem handle 1306 in the direction of arrow C facilitates an increased movement/manipulation of the protruding region PR of the absorber towards the surface S where blood or any other fluid may have collected. Thus, as blood or other fluid collects, the protruding region of the absorber can be brought into contact with the collected blood/fluid by: (i) angularly manipulating the device 1300 and/or (ii) flexing the flexible stem handle 1306 by exerting force on the raised portion 1315 (i.e., in the direction of arrow C) causing the protruding region PR to further flex toward the surface where the blood or fluid is or has collected. The device of FIG. 13 includes a raised portion (i.e., an additional pressure point) on the absorber for using, for example, an index finger to push down and flex/bend the stem in the direction of arrow C, thus bringing the protruding region PR in contact with a surface where blood or other fluid has collected and needs absorption. In use, the thumb and index finger grip 1309 can be gripped using the thumb and second finger adjacent the index finger, while the index finger manipulates the pressure on the raised portion 1315.

In some embodiments the protruding region (PR) and the absorber sponge (AS) of the absorber 1302 include the same absorption characteristic (AC), while according to other embodiments, the protruding region (PR) and the absorber sponge (AS) of the absorber 1302 include different absorption characteristic (i.e., AC1 and AC2).

FIG. 14 illustrates yet another exemplary embodiment of a medical dissector/absorber device 1400 similar to that of FIG. 12, where the dissector 1404 is adhered directly to the absorber region 1402, and the absorber 1402 is adhered to a flexible stem handle 1406. In operation, bending or flexing the flexible stem handle facilitates an increased movement/manipulation of the protruding region PR of the absorber 1402 towards the surface where blood or any other fluid may have collected. Thus, as blood or other fluid collects, the protruding region(s) PR of the absorber 1402 can be brought into contact with the collected blood/fluid by: (i) angularly manipulating the device and/or (ii) flexing the flexible stem handle causing the protruding region PR to further flex toward the surface where the blood or fluid is or has collected.

As depicted by the expanded view 1409 of the protrusion region PR, taken across cross section A-A′ of the absorber 1402, the protrusion region PR includes two protrusion fins or regions 1410a, 1410b (as opposed to a single protrusion). The two protrusion fins or regions 1410a, 1410b facilitate performing an additional absorption action by swiveling the stem 1406 (clockwise or anticlockwise, as indicated by illustrative arrows Ar) in a manner where blood or other liquid is absorbed as a result of the protrusion fins 1410a, 1410b rotatively and thus sequentially brushing over and contacting the blood and/or other liquid.

In some embodiments, both the protrusion fins 1410a, 1410b can include the same absorption properties (i.e., AC). According to other embodiments, each of the protrusion fins 1410a, 1410b can have different absorption properties (i.e., AC1 and AC2), allowing a user or surgeon to rotate the appropriate fin, 1410a or 1410b, into contact with blood and/or other liquid based on the required absorption. For example, if less absorption is needed, the fin with relatively less absorptive properties relative to the other fin is used. Conversely, if more absorption is required, the fin with relatively more absorptive properties relative to the other fin is used.

In the embodiments depicted in FIGS. 11-15, although the absorber and dissector are described and illustrated as being adhered directly to each other, a thin interface (e.g., thin plastic) separating the absorber and dissector by a small amount relative to the embodiment of FIG. 10 may be contemplated. As shown in FIG. 10, the dissector 1004 and absorber 1002 are separated and isolated in part by end plug 1027. In one embodiment, the end plug 1027 may be formed as part of the absorber 1002. Alternatively, the end plug 1027 may include a cavity in which the end of the absorber 1002 fits into. In contrast, for example, the embodiment of FIG. 10 can be adapted for procedures requiring more sheering force by the dissector relative to the embodiments of FIGS. 11-15, whereby the absorbers and dissectors are coupled (e.g., adhered) together directly or via a thin interface. In FIGS. 11-15, too much sheering force by the dissector may transfer to, and compressively deform, the absorber. Thus, depending on how delicate the surgical procedure, different embodiments may be utilized. To transfer less compressive force to the absorber during dissection however, as depicted in FIGS. 11-15, the flexible stem may extend beyond the where the absorber and dissector adhere (i.e., directly, or indirectly), and additionally couples to a portion of the dissector to drive the sheering force.

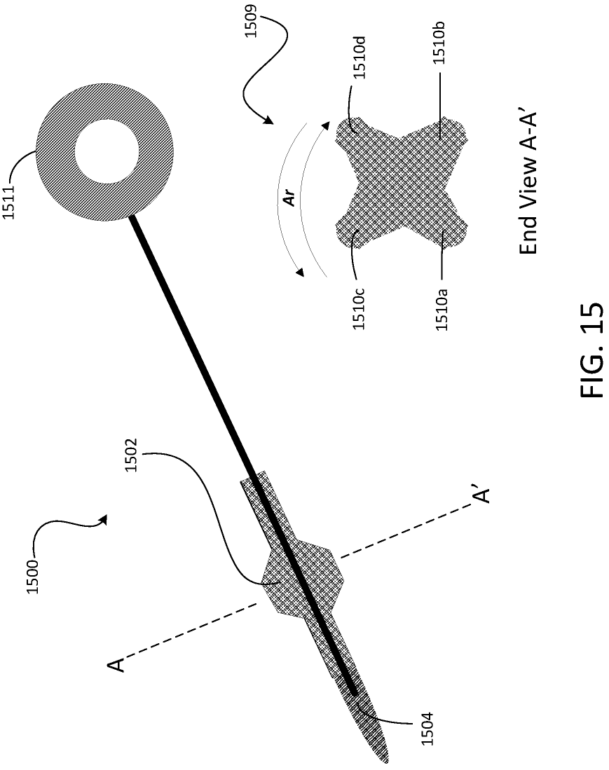

FIG. 15 illustrates yet another exemplary embodiment of a medical dissector/absorber device 1500 similar to that of FIG. 12, where the dissector 1504 is adhered directly to the absorber region 1502, and the absorber 1502 is adhered to a flexible stem handle 1506 having a grip such as a thumb and index finger grip 1511. In operation, bending or flexing the flexible stem handle facilitates an increased movement/manipulation of the protruding region PR of the absorber towards the surface where blood or any other fluid may have collected. Thus, as blood or other fluid collects, the protruding region(s) PR of the absorber 1502 can be brought into contact with the collected blood/fluid by: (i) angularly manipulating the device and/or (ii) flexing the flexible stem handle causing the protruding region PR to further flex toward the surface where the blood or fluid is or has collected.

As depicted by the expanded view 1509 of the protrusion region PR, taken across cross section A-A′ of the absorber 1502, the protrusion region PR includes four protrusion fins or regions 1510a, 1510b, 1510c, 1510d (as opposed to a single protrusion). The two protrusion fins or regions 1510a, 1510b, 1510c, 1510d facilitate performing an additional absorption action by swiveling the stem 1506 (clockwise or anticlockwise, as indicated by illustrative arrows Ar) in a manner where blood or other liquid is absorbed as a result of the protrusion fins 1510a, 1510b, 1510c, 1510d rotatively and thus sequentially brushing over and contacting the blood and/or other liquid.

In some embodiments, the protrusion fins 1510a, 1510b, 1510c, 1510d can include the same absorption properties (i.e., AC). According to other embodiments, each of the protrusion fins can have different absorption properties (i.e., AC1, AC2, AC3, AC4, or any combination thereof), allowing a user or surgeon to rotate the appropriate fin, 1510a, 1510b, 1510c, 1510d, into contact with blood and/or other liquid based on the required absorption. For example, if less absorption is needed, the fin with relatively less absorptive properties relative to the other fin is used. Conversely, if more absorption is required, the fin with relatively more absorptive properties relative to the other fin is used.

For example, fins 1510c and 1510d may include the same absorption characteristic AC1, while fins 1510a and 1510b may include the same absorption characteristic AC2, where AC1 and AC2 have different absorption characteristics. According to another example, fins 1510a, 1510b, 1510c, and 1510d may include absorption characteristic AC1, AC2, AC3, and AC4, respectively, whereby AC1, AC2, AC3, and AC4 all have different absorption characteristics. In other embodiments, for example, fins 1510a, 1510b, 1510c, and 1510d may all include the same absorption characteristic AC.

The devices of the present disclosure can be made of any suitable materials such as surgical stainless steel, titanium, carbon fiber plastic, etc. Also, reference to adhered or adhering in the context of the present disclosure includes any suitable means of coupling such as glue, adhesive, complementary snap-fit assemblies, Velcro, etc.

Absorption characteristics may include any property of a material that describes, among other things, the amount and/or rate at which it can absorb liquid. For example, characteristics such as pores per centimeter (PPC), density, smoothness, solids penetration, water absorption rate, abrasion resistance, squeezability (ease of water expulsion), water release speed, etc., are but some exemplary absorbing characteristics for an absorber.

What is claimed is:

1. A handheld medical dissector/absorber device comprising:
an elongate handle;
a dissector coupled to an end portion of the elongate handle; and
an absorber located at the end portion of the elongate handle and coupled to both the dissector and the elongate handle, wherein the absorber and dissector include a concentric relationship with respect to each other.

2. The device of claim 1, wherein the concentric relationship comprises the absorber and dissector being partially or fully concentric with respect to each other.

3. The device of claim 1, wherein a first portion of the dissector is located concentrically within the absorber and a second portion of the dissector protrudes beyond an end portion of the absorber, and wherein the second portion of the dissector that protrudes beyond the end portion of the absorber is configured to dissect tissue, while the absorber is configured to absorb fluid based on the dissecting of the tissue.

4. The device of claim 1, wherein the absorber comprises a first region including a first absorption characteristic and a second region including a second absorption characteristic, the first absorption characteristic being different relative to the second absorption characteristic.

5. The device of claim 1, further comprising squeezing tongs that are configured to dispense absorbed liquid from the absorber upon actuation of the squeezing tongs to compress the absorber.

6. A handheld medical dissector/absorber device comprising:
an elongate handle having a cavity region;
a dissector coupled to an end portion of the elongate handle; and
an absorber including a first portion concentrically located within the dissector and a second portion located within the cavity region of the elongate handle,
wherein the dissector is configured to dissect tissue, while the first portion of the absorber is exposed and configured to absorb fluid based on the dissecting of the tissue and the second portion of the absorber is configured to absorb the fluid captured by the first portion of the absorber.

7. The device of claim 6, the first portion of the absorber comprises a first channel width and the second portion of the absorber comprises a second channel width that is larger than the first channel width.

8. The device of claim 6, further comprising a suction mechanism coupled to the second portion of the absorber and configured to remove absorbed fluid from the absorber.

9. The device of claim 6, further comprising an actuating lever located on the elongate handle, wherein by actuating the actuating lever towards a portion of the second region of the absorber within the cavity, the portion of the second region is compressed releasing absorbed liquid within the second region of the absorber into the cavity region of the elongate handle portion.

10. The device of claim 9, wherein the elongate handle comprises perforations for emptying the released absorbed liquid from the cavity region.

11. A handheld medical dissector/absorber device comprising:
an elongate handle including a flexible stem;
an absorber including an absorption sponge and a protruding region, the absorption sponge having a top surface coupled to a first portion of the flexible stem and an undersurface from which the protruding region protrudes downwardly; and
a dissector coupled to an end portion of the absorption sponge and a second portion of the flexible stem,
wherein the flexible stem is configured to flex and bring the protruding region in contact with fluid based on a dissection of tissue by the dissector.

12. The device of claim 12, wherein the elongate handle comprises a thumb and index finger grip located at an end of the flexible stem.

13. The device of claim 12, wherein the protruding region of the absorber comprises a same absorption characteristic as the absorbing sponge of the absorber.

14. The device of claim 12, wherein the protruding region of the absorber comprises a first protruding fin and a second protruding fin located adjacent the first protruding fin.

15. The device of claim 14, wherein the first protruding fin and the second adjacent protruding fin comprise a same absorption characteristic.

16. The device of claim 14, wherein the first protruding fin and the second adjacent protruding fin comprise a different absorption characteristic.

17. The device of claim 11, wherein the absorber comprises a PVA sponge that includes Polyvinyl Alcohol.

18. The device of claim 11, wherein the dissector comprises a spun cotton material.
